(19) United States
(12) Patent Application Publication
(10) Pub. No.: US 2025/0369976 A1
(43) Pub. Date: Dec. 4, 2025

(54) AN IMMUNOSENSOR ELECTRODE

(71) Applicant: UNIVERSITI BRUNEI DARUSSALAM, Brunei Darussalam (BN)

(72) Inventors: Minhaz Uddin AHMED, Brunei-Muara (BN); Faheem KAREEM, Karachi (PK); Noor Faizah MOHD-NAIM, Brunei-Muara (BN)

(21) Appl. No.: 19/214,207

(22) Filed: May 21, 2025

(30) Foreign Application Priority Data
May 28, 2024 (BN) BN/N/2024/0055

(51) Int. Cl. G01N 33/68; G01N 27/12; G01N 33/543

(52) U.S. Cl. CPC G01N 33/68; G01N 27/127; G01N 33/5438; G01N 2333/77

(57) ABSTRACT

The present invention is an immunosensor electrode based on a nanocomposite comprising a nanocellulose, a two-dimensional conductive nanomaterial, and a metal oxide nanoparticles to be deposited on a conductive substrate for detecting allergen. The nanocomposite with a large surface area was used as electrochemical mediator and immobilization surface for the antibody to bind with the allergen. The fabricated immunosensor exhibited high selectivity, reproducibility, and interference resistance and achieved excellent recoveries of detecting allergen in real food samples, indicating its potential applicability in food safety monitoring.

### CROSS-REFERENCE TO RELATED APPLICATIONS

This application is based upon and claims the benefit of priority from Brunei Patent Application No. BN/N/2024/0055 filed on May 28, 2024, the contents of which are incorporated herein by reference.

### FIELD OF INVENTION

The present invention relates a nanocomposite and a method of its use for protein detection. More specifically, the present invention relates to an immunosensor electrode and a method of its use for allergen protein detection.

### BACKGROUND OF INVENTION

The basis of immunosensor electrode is the precise recognition of antigen and antibody. One of the most frequent challenges encountered in the construction of an efficient immunosensor electrode is the procedure used for immobilizing the antibodies. The antibodies have been immobilized on sensing surface by using variety of techniques, such as simple physical adsorption, chemical crosslinking, binding to gold-thiol monolayers, avidin-biotin affinity, and entrapment in conducting polymers [Ju, H., G. Lai, and F. Yan, Immunosensing for detection of protein biomarkers. 2017; Dadfar, S. M. M., et al. Adv. Mater. Interfaces, 2021. 8 (10): p. 2002117; Hong, S. P., S. N. A. Zakaria, and M. U. Ahmed, Food Chem. Adv., 2022: p. 100069; Azam, N. F. N., et al., Anal. Sci., 2019. 35 (9): p. 973-978; Purohit, B., et al., Sens. Int., 2020. 1: p. 100040]. According to Wang, H., et al., Anal. Biochem., 2004. 324 (2): p. 219-226 and Abd Manaf, B. A., et al., Surf. Interfaces, 2022: p. 102596], physical adsorption has stability issues and is highly sensitive to environmental changes.

Additionally, other immobilization methods mentioned above have several downsides, including complicated operation, rapid antibody removal, lowered antibody activity, and so forth [Cosnier, S., Biosens. Bioelectron., 1999. 14 (5): p. 443-456.10-12; Chen, H., et al., Int. J. Mol. Sci., 2014. 15 (4): p. 5496-5507; Zacco, E., et al., Biosens. Bioelectron., 2007. 22 (8): p. 1707-1715.]. In comparison to physical adsorption, covalent binding is the favored approach because this method primarily generates a covalent link between modified surface of transducer and protein functional groups with exposed side chains, resulting in an irreversible binding that enables high surface coverage and more effective antibody immobilization. Therefore, it is vital to develop a new covalent linkage-based immobilization approach and explore low-cost matrices for the construction of electrochemical immunosensors.

Nanocellulose has been widely employed as a carrier for the immobilization of biomolecules such as cells, bacteria, enzymes, and antibodies because of its attractive low-cost functional characteristics, biodegradability, and stability. However, due to their poor electrical conductivity and inability to provide an adequate level of sensitivity for biosensor applications, these materials must be modified in order to be used as suitable supporting material in electrochemical biosensing [Mahadeva, S. K., K. Walus, and B. Stoeber, ACS Appl. Mater. Interfaces, 2015. 7 (16): p. 8345-8362; Sun, Q., et al., Biosens. Bioelectron., 2018. 119: p. 237-251]. The numerous reactive hydroxyl groups that nanocellulose has on its surface make it suitable for further chemical modification. Dialdehyde cellulose nanofiber (DCNFs) can be obtained by utilizing sodium metaperiodate as an oxidant to convert vicinal hydroxyl groups of cellulose nanofibers at position 2 and 3 to aldehyde groups [Chen, D. and T. G. van de Ven, Cellulose, 2016. 23 (2): p. 1051-1059.].

Nanostructured metal oxides (NMOs) have been shown to have fascinating electrical and optical properties due to electron confinement, high catalytic efficiency, high aspect ratio, greater surface reaction activity, and strong adsorption capacity [Das, M., et al., Appl. Phys. Lett., 2011. 99 (14): p. 143702]. Zirconia has been proven to possess intriguing physiochemical and biosensing properties among the many NMOs. ZrO₂ has numerous remarkable characteristics that make it a potential material for a transducer in the construction of biosensor, including biocompatibility, superior electrical and surface charge properties, and oxygen moieties [Liu, G. and Y. Lin, Anal. Chem., 2005. 77 (18): p. 5894-5901]. The research findings suggest that Zirconia nanoparticles have a tendency to cluster together and agglomerate. To overcome these challenges, it is necessary to use a substrate material with a large surface area and good conductivity to enhance the biosensing potential of ZrO₂ nanoparticles [Zhao, N., et al., J. Am. Chem. Soc., 2006. 128 (31): p. 10118-10124.]. MXene (Ti₃C₂) is a 2D transition metal carbide, nitride, or carbonitride that has a layered structure. It is part of the 2D family of materials that share this layered structure. It has outstanding electrical conductivities, large surface area, high hydrophilicity, and easy functionalization, which enables it to amplify the electrochemical signals and construct highly efficient electrochemical biosensor [Wei, W., et al., Sens. Actuators, B, 2021. 332: p. 129525; Shao, Y., et al., Microchim. Acta, 2019. 186: p. 1-9.]. The potential application of MXene in sensing and biosensing are still being investigated. Several composites based on MXene, such as MXene/Ag, MXene/magnetic iron oxide, and MXene/TiO₂/C, demonstrated excellent electrocatalytic activity and extraordinary prolonged cycle lifetime [Zhang, Z., et al., ACS Sustainable Chem. Eng., 2016. 4 (12): p. 6763-6771; Zhang, Q., et al., Nanoscale, 2016. 8 (13): p. 7085-7093; Zou, G., et al., ACS Appl. Mater. Interfaces, 2016. 8 (34): p. 22280-22286.]. Consequently, we can say that the composite made of MXene, and zirconia NPs offers a number of noteworthy benefits, including controlled nanostructure, improved catalytic characteristics, and signal amplification.

The prevalence of egg allergy has increased globally during the past several years and is turning into one of the biggest health concerns of modern time [Zou, G., et al., ACS Appl. Mater. Interfaces, 2016. 8 (34): p. 22280-22286]. Egg is one of the most prominent causes of the food allergies in kids, especially those under three years old [Ng, T. W., P. Holt, and S. Prescott, Allergy, 2002. 57 (3): p. 207-214; Caubet, J.-C. and J. Wang, Pediatric Clinics, 2011. 58 (2): p. 427-443]. In some countries, it is regarded as the primary allergen, surpassing even an allergy to cow's milk. Egg allergy symptoms can range from moderate to severe, including diarrhea, itching, vomiting, abdominal pain, stomach ache, and even anaphylactic shock [Tan, J. W. and P. Joshi, J. Pediatr. child health, 2014. 50 (1): p. 11-15]. The most common egg allergens are ovalbumin, ovomucoid, ovotransferrin, and lysozyme. Ova is the primary protein in egg whites and accounts for around 54% of the total protein content. It can be used as a biomarker for egg contamination in foodstuffs [Zeng, Q., X. Huang, and M. Ma, Int. J. Electrochem. Sci, 2017. 12: p. 3965-3981]. Several conventional methods are used for the detection of Ova including enzyme-linked immunosorbent assay (ELISA) [Shon, D.-H., et al., Food Sci. Anim. Resour., 2010. 30 (1): p. 36-41], Western blot [Meyer, P. and S. Zanetti, J. Food Sci., 2015. 80 (9): p. T2102-T2109], capillary electrophoresis [Morani, M., M. Taverna, and T. D. Mai, Electrophoresis, 2019. 40 (18-19): p. 2618-2624], immuno-peroxidase staining [Janssen, F. W., G. Voortman, and J. A. De Baaij, J. Agric. Food. Chem., 1987. 35 (4): p. 563-567], flow cytometry [Ross, G., M. G. Bremer, and M. W. Nielen, Anal. Bioanal. Chem., 2018. 410 (22): p. 5353-5371], and polymerase chain reaction [Alves, R. C., et al., Crit. Rev. Food Sci. Nutr., 2016. 56 (14): p. 2304-2319]. Regardless of the high sensitivity of the currently used analytical approaches, they are very expensive, time-consuming, labor-intensive and demand highly skilled workers. Nowadays, electrochemical biosensors have gained prominence as one of the most intriguing analytical methods and possess the potential in numerous applications, including food quality monitoring and clinical diagnostics [Abd Manaf, B. A., et al., Surf. Interfaces, 2022: p. 102596; Noor Azam, N. F., et al., Microchim. Acta, 2020. 187: p. 1-9; Hong, S. P., S. N. A. Zakaria, and M. U. Ahmed, Food Chem. Adv., 2022: p. 100069; Hong, S. P., et al., Bioelectrochemistry, 2022. 147: p. 108172; Ranjan Srivastava, V., R. Kumari, and P. Chandra, Electroanalysis, 2023. 35 (8): p. e202200355; Suman, P. and P. Chandra, Immunodiagnostic technologies from laboratory to point-of-care testing. 2021: Springer]. The attraction of electrochemical biosensor are their low-cost, easy to use, low detection limits, rapid analysis, excellent sensitivity, selectivity, and affinity [Ramachandran, R., et al., Inorg. Chem. Front., 2019. 6 (12): p. 3418-3439; Rizwan, M., et al., Sens. Actuators, B, 2018. 255: p. 557-563; Nandi, I., S. Rai, and P. Chandra, Talanta, 2023: p. 125124; Borse, V., P. Chandra, and R. Srivastava, BioSensing, Theranostics, and Medical Devices. 2022: Springer.]. To the best of our knowledge, only a limited number of electrochemical biosensors have been published for the detection of the egg allergen ovalbumin [Zeng, Q., X. Huang, and M. Ma, Int. J. Electrochem. Sci, 2017. 12: p. 3965-3981; Khumsap, T., et al., Bioelectrochemistry, 2021. 140: p. 107805; Eissa, S., et al., Analyst, 2013. 138 (15): p. 4378-4384] [28, 44, 45].

### SUMMARY OF INVENTION

Accordingly, the present invention provides a conductive nanocomposite for configuring an electrode of an immunosensor, the conductive nanocomposite comprising a cellulose derivative which is capable of binding with a biological recognition element wherein the biological recognition element having selectivity towards an analyte to be detected using the electrode, a nanostructured metal oxide which is capable of facilitating an electric conductivity of the electrode and a two-dimensional conductive nanomaterial is capable of connecting the cellulose derivative to the nanostructured metal oxide, wherein the conductive nanocomposite facilitates a measurement of the electric conductivity of the electrode upon an interaction between the biological recognition element bound on the cellulose derivative with the analyte.

The present invention further provides an immunosensor electrode of an electrochemical immunosensor system for detecting an egg protein, the immunosensor electrode comprising a conductive nanocomposite formed on a surface of a conductive substrate and a biological recognition element bound onto the conductive nanocomposite wherein the biological recognition element is selective towards the egg protein, wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein.

Also provided is a method for preparing an immunosensor electrode for detecting ovalbumin, wherein the methods comprising steps of (a) dispersing a MXene nanosheet into an aqueous solution to form a MXene suspension, (b) mixing the MXene suspension with a nanocellulose derivative to form a mixture, (c) adding a suspension of zirconium oxide nanoparticles into the mixture to form a nanocomposite homogeneous suspension, (d) drop-casting the nanocomposite homogeneous suspension onto a surface of a conductive substrate to obtain a surface modified conductive substrate, (e) drying the surface modified conductive substrate at a temperature range of 30 to 50° C. for a period between 30 min to 120 min to form a dried nanocomposite on the conductive substrate, (f) treating the dried nanocomposite on the conductive substrate with an anti-Ova solution, (g) washing the treated conductive substrate using the phosphate-buffered saline (PBS) solution to obtain a washed conductive substrate, (h) treating the washed conductive substrate with a bovine serum albumin solution to obtain a bovine serum albumin treated conductive substrate and (i) washing the bovine serum albumin treated conductive substrate using the PBS solution to obtain the immunosensor electrode for detecting ovalbumin.

Further provided is a method of using an immunosensor electrode for detecting ovalbumin, the immunosensor electrode comprising a conductive nanocomposite formed on a surface of a conductive substrate and a biological recognition element bound onto the conductive nanocomposite wherein the biological recognition element is selective towards the egg protein, wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein, wherein the method comprise steps of (a) extracting a food sample by incubating the food sample with a PBS solution at a temperature range of 30 to 60° C. for a period range of a period of 20 min to 24 hours to obtain a food sample mixture, (b) centrifuging the food sample mixture to obtain a supernatant, (c) diluting the supernatant using a second PBS solution with a ratio of range from 1:100 to 1:1000 to obtain a diluted supernatant solution, (d) incubating the diluted supernatant solution for a period of 15 min to 60 min with the immunosensor electrode, (e) detecting the ovalbumin by conducting an electrochemical measurement using the immunosensor electrode and an electrochemical workstation, wherein the electrochemical measurement is cyclic voltammetry (CV) and (f) detecting the presence of ovalbumin by conducting an electrochemical measurement using the immunosensor electrode and an electrochemical workstation, wherein the electrochemical measurement is differential pulse voltammetry (DPV), wherein the immunosensor electrode is used as a working electrode in a three-electrode electrochemical workstation, wherein the concentration of ovalbumin is determined by measuring the DPV using the immunosensor electrode, wherein a low concentration of ovalbumin results in a high conductivity measured by the electrochemical workstation using the immunosensor electrode, and wherein a high concentration of ovalbumin resulted in a low conductivity measured by the electrochemical workstation using the immunosensor electrode.

Last but not least, there is also provided a method of using an immunosensor electrode for detecting ovalbumin, the method comprising steps of (a) dispersing a MXene nanosheet into an aqueous solution to form a MXene suspension, (b) mixing the MXene suspension with a nanocellulose derivative to form a mixture, (c) adding a suspension of zirconium oxide nanoparticles into the mixture to form a nanocomposite homogeneous suspension, (d) drop-casting the nanocomposite homogeneous suspension onto a surface of a conductive substrate to obtain a surface modified conductive substrate, (e) drying the surface modified conductive substrate at a temperature range of 30 to 50° C. for a period between 30 min to 120 min to form a dried nanocomposite on the conductive substrate, (f) treating the dried nanocomposite on the conductive substrate with an anti-Ova solution, (g) washing the treated conductive substrate using the phosphate-buffered saline (PBS) solution to obtain a washed conductive substrate, (h) treating the washed conductive substrate with a bovine serum albumin solution to obtain a bovine serum albumin treated conductive substrate, (i) washing the bovine serum albumin treated conductive substrate using the PBS solution to obtain the immunosensor electrode for detecting ovalbumin, (j) extracting a food sample by incubating the food sample with a PBS solution at a temperature range of 30 to 60° C. for a period range of a period of 20 min to 24 hours to obtain a food sample mixture, (k) centrifuging the food sample mixture to obtain a supernatant, (1) diluting the supernatant using a second PBS solution with a ratio of range from 1:100 to 1:1000 to obtain a diluted supernatant solution, (m) incubating the diluted supernatant solution for a period of 15 min to 60 min with the immunosensor electrode, (n) detecting the ovalbumin by conducting an electrochemical measurement using the immunosensor electrode and an electrochemical workstation, wherein the electrochemical measurement is cyclic voltammetry (CV) and (o) detecting the presence of ovalbumin by conducting an electrochemical measurement using the immunosensor electrode and an electrochemical workstation, wherein the electrochemical measurement is differential pulse voltammetry (DPV), wherein the immunosensor electrode is used as a working electrode in a three-electrode electrochemical workstation, wherein the concentration of ovalbumin is determined by measuring the DPV using the immunosensor electrode, wherein a low concentration of ovalbumin results in a high conductivity measured by the electrochemical workstation using the immunosensor electrode, and wherein a high concentration of ovalbumin resulted in a low conductivity measured by the electrochemical workstation using the immunosensor electrode.

### BRIEF DESCRIPTION OF THE DRAWINGS

FIG. 1 shows the fabrication and detection steps of the electrochemical immunosensor;

FIG. 2A is the FTIR spectrum of modified cellulose nanofiber and dialdehyde cellulose nanofiber;

FIG. 2B is the SEM micrograph MXene nanosheets;

FIG. 2C is the SEM image of ZrO₂ NPs showing the spherical like morphology

FIG. 2D is the SEM micrograph of synthesized nanocomposite (DCNF-MXene@ZrO₂) at 200 nm;

FIG. 2E is the SEM micrograph of synthesized nanocomposite (DCNF-MXene@ZrO₂) at 100 nm;

FIG. 3A is the SEM image of CNF;

FIG. 3B is the SEM micrograph of DCNF;

FIG. 4A is the cyclic voltammogram of different modified electrodes at scan rate of 100 mV/s;

FIG. 4B is the DPV of different proposed electrodes in 5 mM [Fe(CN)₆]³⁻/⁻⁴ at 100 mV/s;

FIG. 4C is the CVs of modified electrodes at a different scan rate of 60, 80, 100, 130, 150, 170, and 190 mV/s;

FIG. 4D is the linear relationship between anodic and cathodic peak currents vs square root of scan rates;

FIG. 5A is the cyclic voltammogram of different modified electrodes in the potential range of −0.2 to +0.8 V at scan rate of 100 mV/s;

FIG. 5B is the DPV curves of various modified electrodes in 5 mM [Fe(CN)₆]³⁻/⁻⁴ at 100 mV/s in the potential range of −0.2 to +0.6 V;

FIG. 6A shows the effects of antibody concentration;

FIG. 6B shows the reaction time of antibody on nanocomposite modified GCE;

FIG. 6C shows the incubation time of antigen on the designed immunosensor;

FIG. 6D shows the effect of various pH on fabricated immunosensor;

FIG. 7A shows the DPV curves in response to different Ova concentration (0.01 to 1000 μg/mL);

FIG. 7B shows the calibration curve of the designed immunosensor between the peak current and logarithm of the Ova concentration in 5.0 mM [Fe(CN)₆]³⁻/⁻⁴, v=100 mV s⁻¹ at solution pH of 7.5;

FIG. 8 shows the reproducibility of designed immunosensor at four different electrodes under optimum experimental conditions;

FIG. 9A shows the stability of the developed biosensor over a period of 15 days; and

FIG. 9B shows the selectivity of designed Ova immunosensor against Ovm (ovomucoid), CN (casein), β-LG (β-lactoglobulin), Gli (gliadin), and mixture of all proteins with Ova (Ova with Inf.).

### DETAILED DESCRIPTION OF THE PREFERRED EMBODIMENTS

Broadly speaking, the present invention provides a conductive nanocomposite for configuring an electrode of an immunosensor, the conductive nanocomposite comprising a cellulose derivative which is capable of binding with a biological recognition element wherein the biological recognition element having selectivity towards an analyte to be detected using the electrode, a nanostructured metal oxide which is capable of facilitating an electric conductivity of the electrode and a two-dimensional conductive nanomaterial is capable of connecting the cellulose derivative to the nanostructured metal oxide, wherein the conductive nanocomposite facilitates a measurement of the electric conductivity of the electrode upon an interaction between the biological recognition element bound on the cellulose derivative with the analyte.

Preferably, the cellulose derivative is selected from a group of nanocellulose, consisting of cellulose nanofibrils, cellulose nanocrystals, or any combinations thereof. In this case, the nanocellulose is modified to obtain a nanocellulose derivate with at least an additional functional group, wherein the at least one additional functional group is selected from a functional group consisting of aldehyde, hydroxyl, amino, carboxyl, thiol, or epoxy groups.

Preferably, the two-dimensional conductive nanomaterial is a MXene or a graphene. In this case, the two-dimensional nanomaterial is a MXene with a general formula of Mₙ₊₁XₙTₓ, wherein M is a transition metal, X is selected from a group consisting of carbon or nitrogen, T is selected from a group of surface functional groups consisting —OH, ═O or —F, and n is an integer from 1 to 3 and the two-dimensional nanomaterial is a MXene with a general formula of Mₙ₊₁XₙTₓ, wherein M is Titanium, X is carbon, T is surface functional groups from a group of —OH, ═O and —F, and n is an integer from 1 to 3.

Preferably, the nanostructured metal oxide is selected from a group consisting of zirconium oxide, zinc oxide (ZnO), titanium dioxide (TiO₂), or nickel oxide (NiO).

Broadly speaking, the present invention provides an immunosensor electrode of an electrochemical immunosensor system for detecting an egg protein, the immunosensor electrode comprising a conductive nanocomposite formed on a surface of a conductive substrate and a biological recognition element bound onto the conductive nanocomposite wherein the biological recognition element is selective towards the egg protein, wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein.

Preferably, the conductive substrate is a glassy carbon electrode, indium tin oxide (ITO), Fluorine-doped tin oxide (FTO) or screen printed electrodes.

Preferably, the cellulose derivative of the nanocomposite is selected from a group of nanocellulose, consisting of cellulose nanofibrils, cellulose nanocrystals or any combination thereof. In this case, the nanocellulose is modified to obtain a nanocellulose derivate with at least an additional functional group, wherein the at least one additional functional group is selected from a functional group consisting of aldehyde, hydroxyl, amino, carboxyl, thiol, or epoxy groups.

Preferably, the two-dimensional conductive nanomaterial is a MXene or a graphene. In this case, the two-dimensional nanomaterial is a MXene with a general formula of Mₙ₊₁XₙTₓ, wherein M is a transition metal, X is selected from a group consisting of carbon or nitrogen, T is selected from a group of surface functional groups consisting —OH, ═O or —F, and n is an integer from 1 to 3 and the two-dimensional nanomaterial is a MXene, with a general formula of Mₙ₊₁XₙTₓ, wherein M is Titanium, X is carbon, T is surface functional groups from a group of —OH, ═O and —F, and n is an integer from 1 to 3.

Preferably, the nanostructured metal oxide is selected from a group consisting of zirconium oxide, zinc oxide (ZnO), titanium dioxide (TiO₂), or nickel oxide (NiO).

Preferably, the egg protein is selected from a group consisting of ovalbumin, ovomucoid, ovotransferrin, ovomucin, avidin, flavoprotein or phosvitin.

Preferably, the biological recognition element is an antibody having selectivity towards the egg protein, the egg protein being selected from a group consisting of ovalbumin, ovomucoid, ovotransferrin, ovomucin, avidin, flavoprotein or phosvitin. In this case, the at least one biological recognition element is an antibody having selectivity towards ovalbumin.

Broadly provided is a method for preparing an immunosensor electrode for detecting ovalbumin, wherein the methods comprising steps of:
a) dispersing a MXene nanosheet into an aqueous solution to form a MXene suspension;
b) mixing the MXene suspension with a nanocellulose derivative to form a mixture;
c) adding a suspension of zirconium oxide nanoparticles into the mixture to form a nanocomposite homogeneous suspension;
d) drop-casting the nanocomposite homogeneous suspension onto a surface of a conductive substrate to obtain a surface modified conductive substrate;
e) drying the surface modified conductive substrate at a temperature range of 30 to 50° C. for a period between 30 min to 120 min to form a dried nanocomposite on the conductive substrate;
f) treating the dried nanocomposite on the conductive substrate with an anti-Ova solution;
g) washing the treated conductive substrate using the phosphate-buffered saline (PBS) solution to obtain a washed conductive substrate;
h) treating the washed conductive substrate with a bovine serum albumin solution to obtain a bovine serum albumin treated conductive substrate; and
i) washing the bovine serum albumin treated conductive substrate using the PBS solution to obtain the immunosensor electrode for detecting ovalbumin.

Preferably, step (a) comprises ultrasonicating 50 mg MXene nanosheets in 10 ml water to obtain a uniform MXene suspension.

Preferably, step (b) comprises sonicating the mixture for 15 minutes.

Preferably, step (b) comprises modifying a nanocellulose using a periodate oxidation method to obtain a dialdehyde nanocellulose. In this case, the periodate oxidation method comprises treating the nanocellulose with a periodate salt for 4-72 hours at a temperature range of 30 to 80° C. in absence of light and the periodate salt is sodium metaperiodate or sodium orthoperiodate.

Preferably, the suspension of zirconium oxide nanoparticles in step (c) is obtained by dispersing a nanostructured zirconium oxide powder in distilled water.

Preferably, the homogenous nanocomposite suspension in step (c) is subjected to further mixing and sonication for a period range of 30 min to 120 min.

Preferably, the conductive substrate is a glassy carbon electrode, indium tin oxide (ITO), Fluorine-doped tin oxide (FTO) or screen printed electrodes.

Preferably, step (f) is carried out by using 4 μl of 1 μg/ml of the anti-Ova solution.

Preferably, step (f) comprises incubating the treated conductive substrate with the anti-Ova solution at a temperature range of 25 to 35° C. for 1 hour.

Preferably, step (h) comprises dropping 10 μL of 0.1% bovine serum albumin solution onto the surface of the washed conductive substrate and incubating the bovine serum albumin treated conductive substrate at 25° C. for 45 min to block the non-reactive sites and to reduce non-specific interactions on the electrode surface.

Broadly, there is a provided is a method of using an immunosensor electrode for detecting ovalbumin, the immunosensor electrode comprising a conductive nanocomposite formed on a surface of a conductive substrate and a biological recognition element bound onto the conductive nanocomposite wherein the biological recognition element is selective towards the egg protein, wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein, wherein the method comprise steps of:
a) extracting a food sample by incubating the food sample with a PBS solution at a temperature range of 30 to 60° C. for a period range of a period of 20 min to 24 hours to obtain a food sample mixture;
b) centrifuging the food sample mixture to obtain a supernatant;
c) diluting the supernatant using a second PBS solution with a ratio of range from 1:100 to 1:1000 to obtain a diluted supernatant solution;
d) incubating the diluted supernatant solution for a period of 15 min to 60 min with the immunosensor electrode;
e) detecting the ovalbumin by conducting an electrochemical measurement using the immunosensor electrode and an electrochemical workstation, wherein the electrochemical measurement is cyclic voltammetry (CV); and
f) detecting the presence of ovalbumin by conducting an electrochemical measurement using the immunosensor electrode and an electrochemical workstation, wherein the electrochemical measurement is differential pulse voltammetry (DPV)
wherein the immunosensor electrode is used as a working electrode in a three-electrode electrochemical workstation,
wherein the concentration of ovalbumin is determined by measuring the DPV using the immunosensor electrode,
wherein a low concentration of ovalbumin results in a high conductivity measured by the electrochemical workstation using the immunosensor electrode, and
wherein a high concentration of ovalbumin resulted in a low conductivity measured by the electrochemical workstation using the immunosensor electrode.

Broadly, the present invention is a method of using an immunosensor electrode for detecting ovalbumin, the method comprising steps of:
(a) dispersing a MXene nanosheet into an aqueous solution to form a MXene suspension;
(b) mixing the MXene suspension with a nanocellulose derivative to form a mixture;
(c) adding a suspension of zirconium oxide nanoparticles into the mixture to form a nanocomposite homogeneous suspension;
(d) drop-casting the nanocomposite homogeneous suspension onto a surface of a conductive substrate to obtain a surface modified conductive substrate;
(e) drying the surface modified conductive substrate at a temperature range of 30 to 50° C. for a period between 30 min to 120 min to form a dried nanocomposite on the conductive substrate;
(f) treating the dried nanocomposite on the conductive substrate with an anti-Ova solution;
(g) washing the treated conductive substrate using the phosphate-buffered saline (PBS) solution to obtain a washed conductive substrate;
(h) treating the washed conductive substrate with a bovine serum albumin solution to obtain a bovine serum albumin treated conductive substrate;
(i) washing the bovine serum albumin treated conductive substrate using the PBS solution to obtain the immunosensor electrode for detecting ovalbumin;
g) extracting a food sample by incubating the food sample with a PBS solution at a temperature range of 30 to 60° C. for a period range of a period of 20 min to 24 hours to obtain a food sample mixture;
h) centrifuging the food sample mixture to obtain a supernatant;
i) diluting the supernatant using a second PBS solution with a ratio of range from 1:100 to 1:1000 to obtain a diluted supernatant solution;
j) incubating the diluted supernatant solution for a period of 15 min to 60 min with the immunosensor electrode;
k) detecting the ovalbumin by conducting an electrochemical measurement using the immunosensor electrode and an electrochemical workstation, wherein the electrochemical measurement is cyclic voltammetry (CV); and
l) detecting the presence of ovalbumin by conducting an electrochemical measurement using the immunosensor electrode and an electrochemical workstation, wherein the electrochemical measurement is differential pulse voltammetry (DPV),
wherein the immunosensor electrode is used as a working electrode in a three-electrode electrochemical workstation,
wherein the concentration of ovalbumin is determined by measuring the DPV using the immunosensor electrode,
wherein a low concentration of ovalbumin results in a high conductivity measured by the electrochemical workstation using the immunosensor electrode, and
wherein a high concentration of ovalbumin resulted in a low conductivity measured by the electrochemical workstation using the immunosensor electrode.

The present invention is now explained in detail in the below paragraphs.

### Materials and Reagents

Anti-Ovalbumin monoclonal antibody, Ovalbumin from chicken egg white, bovine serum albumin (BSA), zirconium oxide (ZrO₂) nanopowder, potassium ferrocyanide (K₄[Fe(CN₆)]), potassium ferricyanide (K₃[Fe(CN₆)]), potassium chloride, sodium chloride, sodium phosphate monobasic, sodium phosphate dibasic, sodium metaperiodate, β-lactoglobulin, casein, gliadin was all purchased from Sigma-Aldrich (Saint Louis, USA). Other chemicals such as ovomucoid were purchased from Lifespan Bioscience, Inc. (Washington, USA). Ti₃C₂Tₓ (MXene) multilayer was purchased from Nanochemazone (Alberta, Canada). Cellulose nanofiber (10-20 nm wide, 2-3 μm length) was purchased from Nanografi (Ankara, Turkey). Standard solutions of protein and antibody were prepared in phosphate buffer saline (PBS) (10 mM, pH 7.4). In all sample preparations, distilled water from the water purification system (18 MΩ, Milli-Q) was used.

### Instrumentation

The electrochemical measurements including cyclic voltammetry (CV) and differential pulse voltammetry (DPV) were carried out with an AUTOLAB modular Potentiostat/Galvanostat (Mehtrohm Autolab B.V., Utrecht, The Netherlands) linked to a personal desktop with NOVA software version 2.1. Ultrasonication and pH measurements were performed using FB 15046 Fisherbrand™ and Thermo Scientific™ Eutech™ pH 700 Meter. For the morphology of nanomaterials, a Field Emission Scanning Electron Microscope (FE-SEM) JSM-7610F (JEOL ltd., Tokyo, Japan) was utilized. Each sample was carbon-coated for 30 seconds prior to FE-SEM analysis and analyzed in secondary electron imaging (SEI) at a voltage of 10 kV and 10,000-100,000 magnifications. The present invention employed a standard three-electrode configuration that comprised a glassy carbon working electrode, Ag/AgCl reference electrode, and a platinum wire auxiliary electrode. All experiments were conducted at room temperature, and the error bars on the graph represent the standard deviation of at least three replicates (n=3) for each experiment.

### Synthesis of Dialdehyde Cellulose Nanofiber

0.4 g of sodium metaperiodate (NaIO₄) was added into 25 mL of 0.6 wt % CNF solution and heated to 60° C. for 12 h in dark while stirring continuously. Aluminum foil was placed over the reaction flask to avoid photo-induced decomposition of periodate. After 12 h, the reaction was stopped and the dialdehyde cellulose nanofiber was obtained by repeatedly washing with distilled water.

### Preparation of DCNF-MXene@ZrO₂ Nano Framework

First, 5 mg/mL of MXene stock solution was prepared in distilled water by adding 50 mg of MXene nanosheets in 10 mL of distilled water and then ultrasonically treated for a period of time to get a uniform suspension. After that, calculated volume of optimized amount (0.25 mg/mL) of MXene solution were mixed with the 0.01% of DCNF solution and sonicated for 15 min. Similarly, a stock solution of ZrO₂ NPs was prepared in distilled water and known volume of optimized amount (0.25 mg/mL) of this solution was mixed with 0.01% DCNF solution and sonicated for 15 min. A known volume of MXene, ZrO₂ and DCNF solutions were mixed to form the nano framework (DCNF-MXene@ZrO₂), which will have the final concentration of 0.01% for DCNF and 0.25 mg/mL for MXene and zirconium oxide NPs. The nano framework stirred for 30 min followed by 45 min sonication to obtain a homogenous suspension. The prepared nanocomposite was kept at 4° C. until further use.

### Extraction of Real Food Samples

In order to make homogeneous mixture, 0.1 g of real food samples including egg-free cake, chocolate, chocolate malt powder, and oats were blended and mixed in 1 mL of PBS buffer. The mixtures were then incubated at 40° C. for one hour, and then centrifuged at 20,000 g for 15 minutes. The clear supernatants were then collected and diluted to 1:100 in 10 mM PBS buffer and spiked with 100 μg/mL of Ova Afterwards, the immunosensor was incubated with spiked extracts for 60 min at room temperature, and the DPV responses were recorded.

### Construction of the Designed Immunosensor

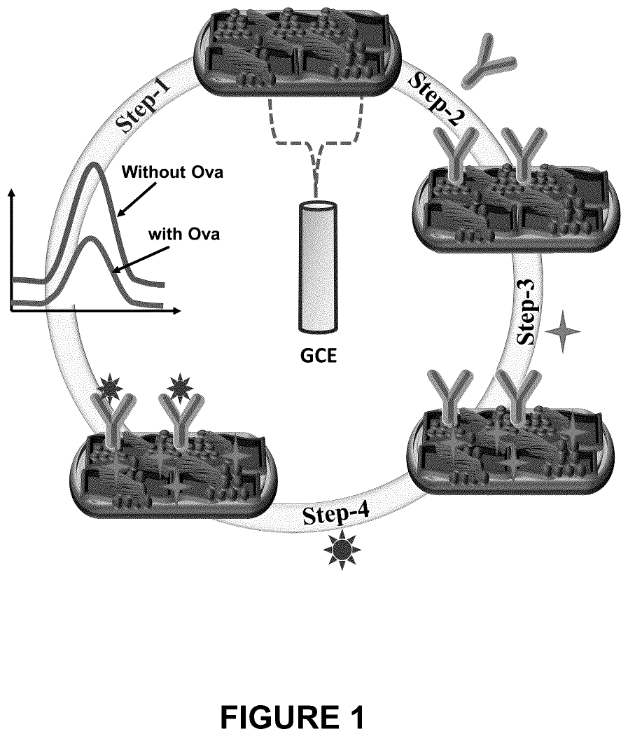

In order to fabricate the electrochemical immunosensor, the bare GCE was polished multiple times with alumina slurry, rinsed with deionized water, and sonicated in an ultrasonic water bath for 3 min as illustrated in FIG. 1. The electrode was then rinsed once more and allowed to air dry. A drop-casting method was used to construct Ova-immunosensor. Firstly, 6 μL of nano framework (NF) was cast on to the surface of GCE to fabricate DCNF-MXene@ZrO₂/GCE and then incubated for 30 min at 40° C. The modified electrode was then treated with 4 μL of 1 μg/mL of anti-Ova solution and incubated for 1 h at room temperature, followed by washing with PBS to eliminate any unwanted physical adsorption. Next, 10 μL of 0.1% BSA was dropped onto the surface of anti-Ova/DCNF-MXene@ZrO₂/GCE and incubated at 25° C. for 45 min to block the non-reactive sites and to reduce non-specific interactions on the electrode surface. After washing with PBS, BSA/anti-Ova/DCNF-MXene@ZrO₂/GCE immunosensor was obtained. For Ova detection, 4 μL of ovalbumin with various concentration was incubated on the fabricated working electrode for 1 h at room temperature and the electrochemical measurements were carried out in 0.01 M PBS (pH 7.4) containing 5 mM [Fe(CN)₆]³⁻/⁻⁴ and 0.1 M KCl. CV and DPV measurements were performed with a potential range between-0.2 to +0.8 and 0.2 to +0.6 V at 100 mV/s scan rate.

### Results and Discussion

### Morphology and Structural Characterization

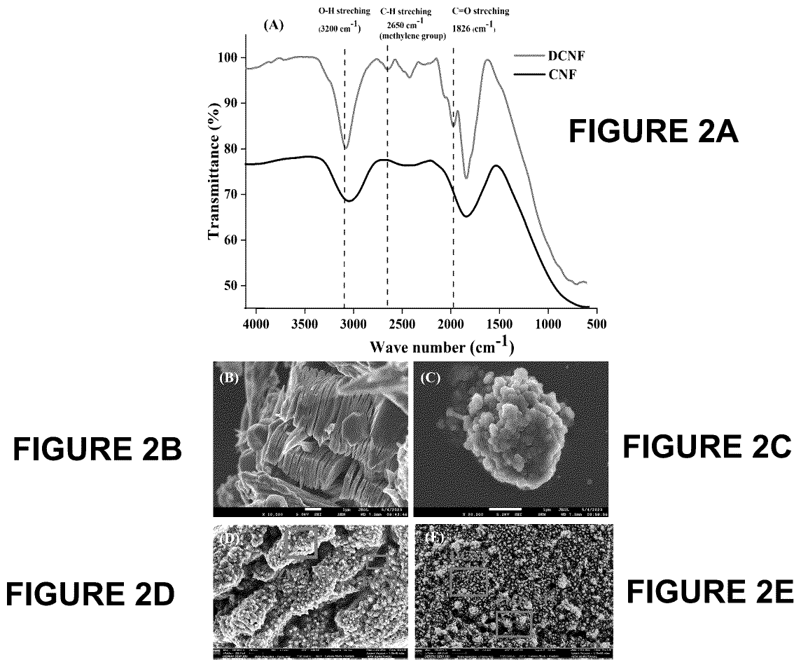

FTIR analysis was carried out to examine the structural changes of CNF during oxidation and the results are shown in FIG. 2A. A new absorption band was remarked at 1750 cm⁻¹ that corresponded to the characteristic absorption peak of aldehyde group (C═O) of DCNF, which suggested that the hydroxyl groups on the surface of CNFs were converted to aldehyde groups. The absorption band appeared at 3100 cm⁻¹ is due to the O—H stretching vibrations whilst the band near 2650 cm⁻¹ is due to methylene group in DCNF. The peak at 1650 cm⁻¹ represented the hydroxyl bending vibration in the adsorbed water.

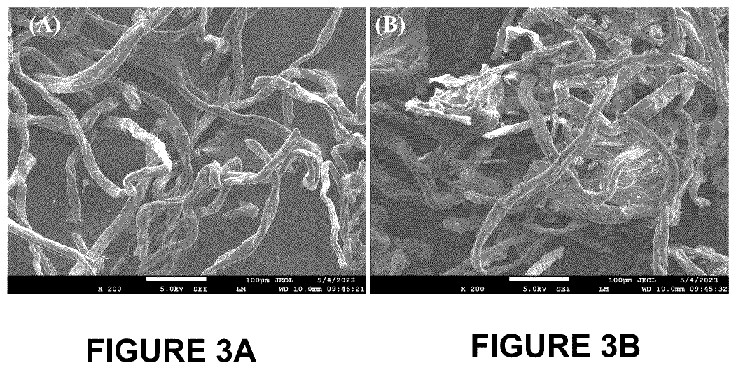

The morphology of modified and unmodified CNFs is shown in FIGS. 3A and 3B. The SEM image clearly demonstrates the long flexible network structure of both NCs. Except for the small curling that was seen in DCNF, there was no discernible difference between them, indicating that the modification process had little impact on the CNF morphologies, wherein the CNF becomes flexible, bends, and even shortens. FIGS. 2B and C show the surface morphology of MXene and ZrO₂ NPs. It is evident that MXene has a multi-layered stacked architecture whilst zirconium oxide NPs are spherical in shape. The morphology of the nano framework (DCNF-MXene@ZrO₂) and its effective synthesis have been examined with FESEM (FIGS. 2D and E). ZrO₂ nanoparticles with a spherical shape are distributed uniformly across the surface of the multilayered MXene nanosheets, whereas the short, flexible fibres are found in gaps between the layers of MXene nanosheets. These results demonstrate the effective synthesis of a nanocomposite material. For better understanding of the nanocomposite morphology, SEM images are provided at two distinct scales (100 and 200 nm).

### Electrochemical Characterization of Nano Framework Modified Electrodes

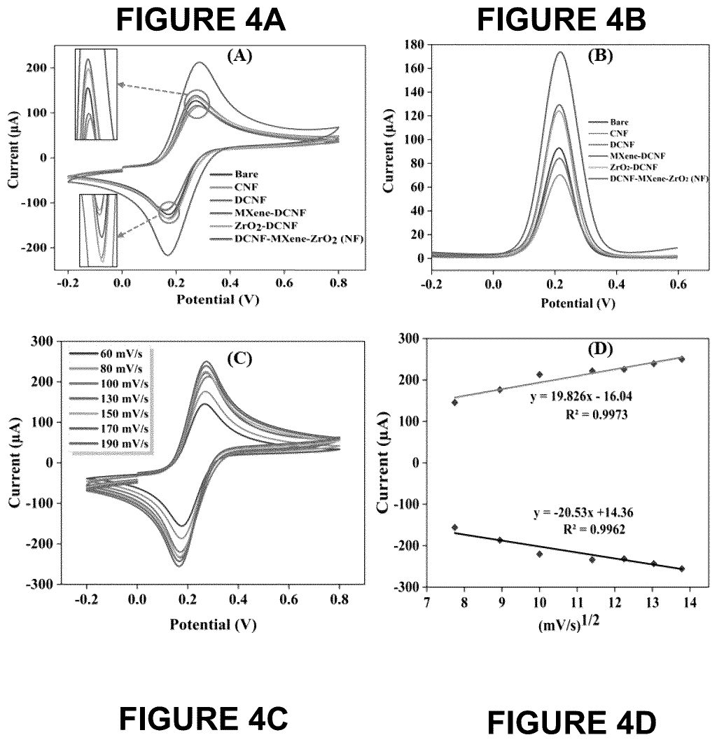

The CV and DPV were used to examine the electrochemical performance of NF modified GCE in 5 mM [Fe(CN)₆]³⁻/⁻⁴ and 0.1 M KCl at a scan rate of 100 mv/s. The CV and DPV curves depicted in FIGS. 4A and B demonstrate a substantial decline in the current response of the GCE in its pristine state subsequent to the application of CNFs and DCNFs via drop-casting onto the electrode surface. The decrease in the current response can be ascribed to the non-conductive characteristics of the nanocellulosic materials, which lead to the obstruction of the glassy carbon electrode surface. The current response was higher in DCNF-modified GCE compared to CNF because the dialdehyde product of CNFs has the improved redox property with abundant aldehyde groups. Additionally, DCNF has increased reactivity caused by the smaller morphology and weaker inter or intra-hydrogen bond. After modification with MXene and ZrO₂ NPs, the current was much higher than that of bare GCE, which could be ascribed to the unique electrical characteristics of MXene and nanostructure zirconia. However, the DCNF-MXene@ZrO₂/GCE exhibited a significantly greater peak current compared to other modified electrodes. This can be attributed to its superior electrical conductivity, substantial surface area, and synergistic interactions between nanostructure zirconia and MXene nanosheet. These interactions accelerated the rate of electron transfer, resulting in an overall enhanced sensing response.

In order to better understand the electrochemical mechanism, the relationship between scan rate and redox probe was investigated. FIG. 4C illustrates the CV of nano-framework modified GCE at various scan rates between 60 and 190 mV/s. As can be seen, increasing the scan rate resulted in higher anodic and cathodic peak currents. Additionally, the redox peak currents exhibited an excellent linear relationship with the square root of scan rates (V^{1/2}) in FIG. 4D, suggesting that the diffusion process was dominated by the interaction between nano framework modified GCE and redox probe.

The electroactive surface area of the proposed electrodes was calculated to be 0.115, 0.122, 0.125, and 0.192 cm² for bare, ZrO₂/GCE, MXene/GCE, and DCNF-MXene@ZrO₂/GCE. These findings indicated that the electroactive surface area could be significantly increased by adding MXene nanosheets and ZrO₂ NPs.

### Electrochemical Characterization of Fabricated Ova Immunosensor

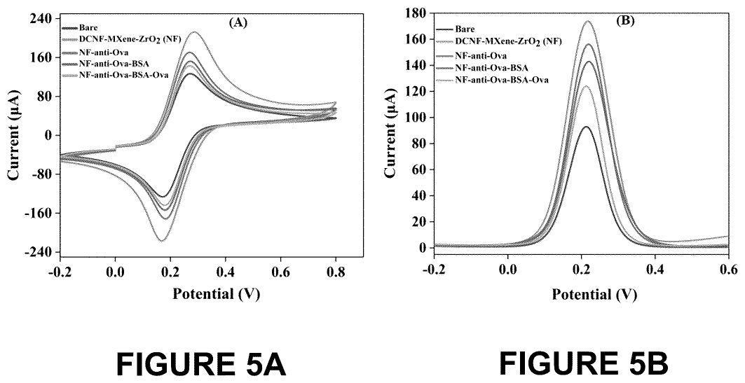

To assess the fabrication process and sensing interface characteristics of designed immunosensor, CV and DPV were employed (FIGS. 5A and 4B). Compared to the bare electrode, the current response of DCNF-MXene@ZrO₂/GCE (NF-modified GCE) was much higher than that of all other modified electrodes because of the synergistic interactions between nanostructure zirconia and MXene nanosheet, as well as their superior electrical conductivity. When Ova antibody was immobilized, the anodic and cathodic current reduced due to the blocking effect imposed by the binding of significant number of Ova to the binding site on the surface of nanocomposite. These findings revealed that anti-Ova was successfully immobilized on the nano framework. Furthermore, the electrochemical signal continuously decreases upon modification with BSA, indicating that BSA effectively adhered to the non-specific reaction sites. When 100 μg/mL of Ova protein was introduced to the biosensor (denoted as Ova/BSA/anti-Ova/DCNF-MXene@ZrO₂/GCE), the peak current was further decreased. The observed phenomenon is caused by the accumulation of protein layers on the electrode surface, hindering the electron transfer across the electrode interface. The consistency between the CV outcomes and DPV results (FIG. 5B) provides support for the successful construction of the immunosensor.

### Optimization of Working Conditions for Ova Immunosensor

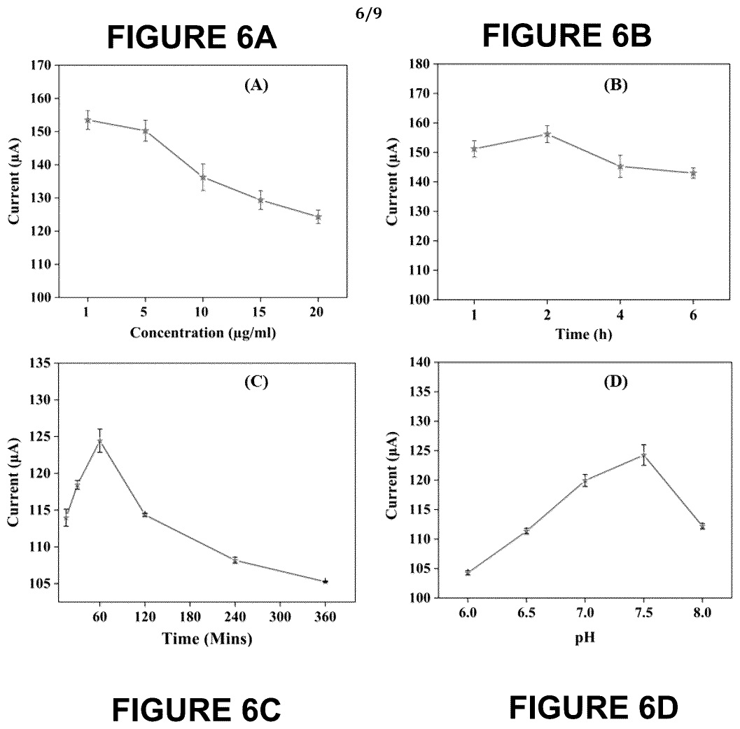

To improve the electrochemical performance of ova immunosensor, four key parameters including antibody concentration and binding time, solution pH and antigen incubation time were optimized. As observed in FIG. 6A, the current response decreased with increasing antibody concentration. However, the peak current did not significantly decrease when the antibody concentration exceeds 10 μg/mL. This observation may be attributed to the saturation of active sites available for anti-Ova immobilization. Therefore, 1 μg/mL was selected as the optimum antibody concentration. The effect of the reaction time of anti-Ova on DCNF-MXene@ZrO₂/GCE was evaluated (FIG. 6B). The current response decreased with increasing immobilization periods up to 2 h, indicating that the binding sites for anti-Ova were saturated. Thus, the incubation time of 2 h was chosen for the subsequent assay. Likewise, the incubation time of Ova on the BSA/anti-Ova/DCNF-MXene@ZrO₂/GCE surface was evaluated. FIG. 6C demonstrates that the DPV signals increase initially as incubation time increases from 15 to 60 minutes before gradually declining after 60 minutes. The peak current showed a stable value, indicating that the biorecognition reaction was complete. Therefore, an incubation period of 60 minutes was selected for Ova in the present invention. In addition, the effect of pH was also investigated in PBS at different pH levels from 6.0 to 8.0. As shown in FIG. 6D, the current response increased significantly when pH changed from 6.0 to 7.5 and then decreased under higher pH condition. This might be due to the fact that proteins exhibited high activity in neutral medium, while the activity of biomolecules was disrupted and diminished in high alkalinity or acidity. Therefore, a pH of 7.5 was selected for the subsequent study.

### Analytical Performance of Fabricated Immunosensor

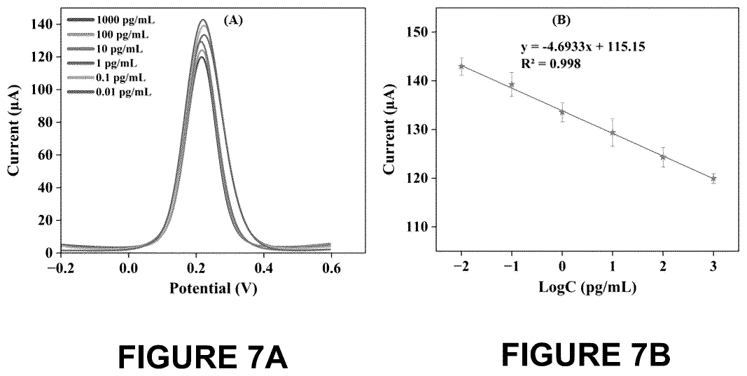

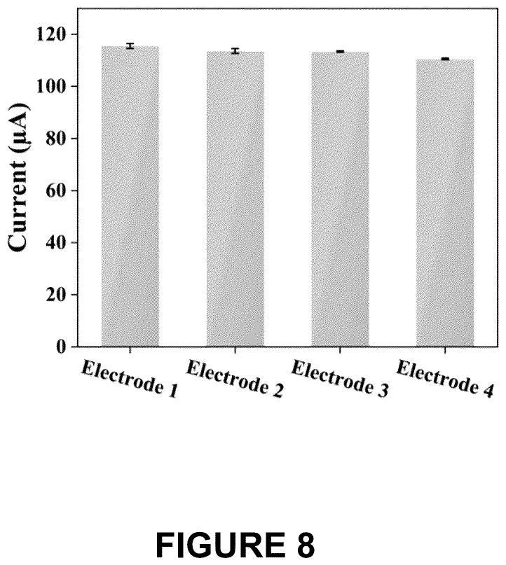

Under optimal working conditions, various concentrations of ovalbumin ranging from 0.01-1000 pg/mL were incubated with the developed immunosensor. The binding between ovalbumin and the antibody-bound electrode was evaluated by the DPV method, and the change in peak current of [Fe(CN)₆]^{3-/-4} was examined. The redox probe's access to the electrode surface, including the addition of bulky protein or negatively charged groups, would slow the charge transfer and, therefore, reduce the current. As demonstrated in FIG. 7A, the DPV peak current decreased as the concentration of Ova increased. The protein's bulky size sterically hinders [Fe(CN)₆]^{3-/-4} anions from accessing the electrode surface, resulting in this reduction in peak current. Moreover, ovalbumin has an electric point of 4.6, which is negatively charged at pH 7.4, causing the repulsion of redox probe anions. As depicted in FIG. 7B, good linearity in the range of 0.01-1000 μg/mL was observed in the plot of the response versus the logarithm of the Ova concentration. The linear regression equation was y=−4.6933x+115.15, with correlation coefficient R²=0.998, with a LOD, LOQ and sensitivity of 1.1 fg/mL, 0.01 pg/mL and 0.1497 μA pg/mL cm⁻². The wide dynamic range and lower LOD revealed that the proposed immunosensor could detect Ova effectively. The modified GCE for the detection of Ova by using nanocellulose/MXene/ZrO₂ nano framework. The modified GCE demonstrated a 197% increase in the electrochemical signal and larger surface area which allowed the Ova detection in the range of 0.01 pg/mL to 1000 μg/mL. The lower limit of detection of DPV biosensor depends on the type of nanomaterials used to modify the electrode for the fabrication of biosensor. Repeatability and long-term stability are key indicators to assess the effectiveness of proposed Ova immunosensor. The electrode-to-electrode repeatability was determined with four different electrodes in the presence of 100 μg/mL Ova. As shown in FIG. 8, the relative standard deviation was estimated to be 2.0%, demonstrating that the fabricated immunosensor exhibited high reproducibility.

### Repeatability, Stability, and Selectivity

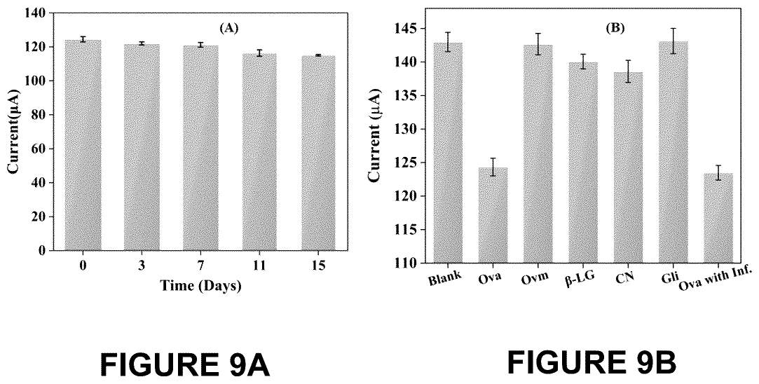

The stability of the immunosensor has been assessed by storing the Ova biosensor at 4° C. for a specified period of time (3, 7, 11 and 15 days). After 3 days of storage, the fabricated biosensor retained about 98% of its original signal. After 15 days of storage at 4° C. the peak current of the Ova immunosensor remained about 92%, proving that the constructed immunosensor had satisfactory stability (FIG. 9A).

To explore the selectivity of the developed immunosensor, 100 μg/mL of different food allergen proteins, namely ovomucoid, casein, β-lactoglobulin, gliadin, and mixture of all proteins with ovalbumin were detected under the same working conditions. The huge difference in DPV peak current between ovalbumin and other proteins indicated that Ova was highly selective in the proposed immunosensor (FIG. 9B). An immunosensor having high selectivity is comparable to that of the blank immunosensor, in this case BSA/anti-Ova/DCNF-MXene@ZrO₂/GCE, with peak current around 143 μA. As result, these interfering proteins show a higher DPV signal than the target protein since the fabricated immunosensor (BSA/a DCNF-MXene@ZrO₂/GCE) does not bind to the interfering substances (non-target proteins).

### Real Sample Analysis

Accurate detection of Ova in food samples is crucial for an effective management strategy of food allergy. Thus, a recovery experiment was carried out to demonstrate the practicability and efficacy of constructed immunosensor in real food samples. The experimental findings, which are summarized in Table 1, showed that the proposed immunosensor were able to detect the presence of Ova allergen with minimal background interferences with excellent recoveries and RSD ranging from 95.48 to 103.74% and 1.16-2.4% for 1:100 and 1:1000 dilution factor, respectively. The present invention provides compelling evidence that the newly developed immunosensor demonstrates negligible interference with the food matrix, and thus holds great promise for detecting Ova allergen in a wide range of practical samples.

TABLE 1
Real sample analysis with proposed method.

| Dilution factor (1:100) | | | | Dilution factor (1:1000) | | | |
|---|---|---|---|---|---|---|---|
| | Spiked | Detected | | | Detected | | |
| | concentration | Concentration | Recovery | RSD | Concentration | Recovery | RSD |
| Samples | (pg/ml) | (pg/ml | (%) | (%) | (pg/ml) | (%) | (%) |
| Egg-free | 100 | 97.45 | 97.45 | 1.16 | 100.38 | 100.38 | 1.38 |
| cake | | | | | | | |
| Milo | 100 | 97.82 | 97.82 | 1.77 | 98.51 | 98.51 | 1.87 |
| Chocolate | 100 | 99.74 | 99.74 | 1.74 | 101.51 | 101.51 | 1.6 |
| Oats | 100 | 95.48 | 95.48 | 2.4 | 103.74 | 103.74 | 2.24 |

In summary, a new nanocomposite is synthesized using nanocellulose, MXene, and ZrO₂ NPs to modify the GCE. This enabled the direct immobilization of antibodies on the aldehyde group of cellulose nanofibers, resulting in highly sensitive and label-free detection of ovalbumin. Owing to the excellent conductive properties of DCNF-MXene@ZrO₂ nano framework, the fabricated GCE showed a 197% increase in the electrochemical signal and a higher surface area than bare electrode, enabling anti-ova immobilization with a greater efficacy at a biofriendly interface. The proposed Ova immunosensor not only showed a wide linear range with a lower LOD, higher sensitivity, but also exhibited outstanding stability, comparable electrochemical response, and good repeatability. These outcomes also coincided with the exceptional recovery rates of the real food samples (95.48-103.74%). This invention expanded the applications of MXene and nanocellulose in the field of biosensors and explored a possible pathway for the construction of MXene-nanocellulose based biosensors for the detection of food allergens.

1. A conductive nanocomposite for configuring an electrode of an immunosensor, the conductive nanocomposite comprising a cellulose derivative which is capable of binding with a biological recognition element wherein the biological recognition element having selectivity towards an analyte to be detected using the electrode, a nanostructured metal oxide which is capable of facilitating an electric conductivity of the electrode and a two-dimensional conductive nanomaterial is capable of connecting the cellulose derivative to the nanostructured metal oxide, wherein the conductive nanocomposite facilitates a measurement of the electric conductivity of the electrode upon an interaction between the biological recognition element bound on the cellulose derivative with the analyte.

2. The conductive nanocomposite as claimed in claim 1, wherein the cellulose derivative is selected from a group of nanocellulose, consisting of cellulose nanofibrils, cellulose nanocrystals or any combinations thereof.

3. The conductive nanocomposite as claimed in claim 2, wherein the nanocellulose is modified to obtain a nanocellulose derivate with at least an additional functional group, wherein the at least one additional functional group is selected from a functional group consisting a of aldehyde, hydroxyl, amino, carboxyl, thiol, or epoxy groups.

4. The conductive nanocomposite as claimed in claim 1, wherein the two-dimensional conductive nanomaterial is a MXene or a graphene.

5. The conductive nanocomposite as claimed in claim 4, wherein the two-dimensional nanomaterial is a MXene with a general formula of Mₙ₊₁XₙTₓ, wherein M is a transition metal, X is selected from a group consisting of carbon or nitrogen, T is selected from a group of surface functional groups consisting —OH, ═O or —F, and n is an integer from 1 to 3.

6. The conductive nanocomposite as claimed in claim 5, wherein the two-dimensional nanomaterial is a MXene with a general formula of Mₙ₊₁XₙTₓ, wherein M is Titanium, X is carbon, T is surface functional groups from a group of —OH, ═O and —F, and n is an integer from 1 to 3.

7. The conductive nanocomposite as claimed in claim 1, wherein the nanostructured metal oxide is selected from a group consisting of zirconium oxide, zinc oxide (ZnO), titanium dioxide (TiO₂), or nickel oxide (NiO).

8. An immunosensor electrode of an electrochemical immunosensor system for detecting an egg protein, the immunosensor electrode comprising a conductive nanocomposite formed on a surface of a conductive substrate and a biological recognition element bound onto the conductive nanocomposite wherein the biological recognition element is selective towards the egg protein,
wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein.

9. The immunosensor electrode as claimed in claim 8, wherein the conductive substrate is a glassy carbon electrode, indium tin oxide (ITO), Fluorine-doped tin oxide (FTO) or screen printed electrodes.

10. The immunosensor electrode as claimed in claim 8, wherein the cellulose derivative of the nanocomposite is selected from a group of nanocellulose, consisting of cellulose nanofibrils, cellulose nanocrystals or any combination thereof.

11. The immunosensor electrode as claimed in claim 10, wherein the nanocellulose is modified to obtain a nanocellulose derivate with at least an additional functional group, wherein the at least one additional functional group is selected from a functional group consisting a of aldehyde, hydroxyl, amino, carboxyl, thiol, or epoxy groups.

12. The immunosensor electrode as claimed in claim 8, wherein the two-dimensional conductive nanomaterial is a MXene or a graphene.

13. The immunosensor electrode as claimed in claim 8, wherein the two-dimensional nanomaterial is a MXene with a general formula of Mₙ₊₁XₙTₓ, wherein M is a transition metal, X is selected from a group consisting of carbon or nitrogen, T is selected from a group of surface functional groups consisting —OH, ═O or —F, and n is an integer from 1 to 3.

14. The immunosensor electrode as claimed in claim 8, wherein the two-dimensional nanomaterial is a MXene, with a general formula of Mₙ₊₁XₙTₓ, wherein M is Titanium, X is carbon, T is surface functional groups from a group of —OH, ═O and —F, and n is an integer from 1 to 3.

15. The immunosensor electrode as claimed in claim 8, wherein the nanostructured metal oxide is selected from a group consisting of zirconium oxide, zinc oxide (ZnO), titanium dioxide (TiO₂), or nickel oxide (NiO).

16. The immunosensor electrode as claimed in claim 8, wherein the egg protein is selected from a group consisting of ovalbumin, ovomucoid, ovotransferrin, ovomucin, avidin, flavoprotein or phosvitin.

17. The immunosensor electrode as claimed in claim 8, wherein the biological recognition element is an antibody having selectivity towards the egg protein, the egg protein being selected from a group consisting of ovalbumin, ovomucoid, ovotransferrin, ovomucin, avidin, flavoprotein or phosvitin.

18. The immunosensor electrode as claimed in claim 8, wherein the at least one biological recognition element is an antibody having selectivity towards ovalbumin.

19. A method for preparing an immunosensor electrode for detecting ovalbumin, wherein the methods comprising steps of:
a) dispersing a MXene nanosheet into an aqueous solution to form a MXene suspension;
b) mixing the MXene suspension with a nanocellulose derivative to form a mixture;
c) adding a suspension of zirconium oxide nanoparticles into the mixture to form a nanocomposite homogeneous suspension;
d) drop-casting the nanocomposite homogeneous suspension onto a surface of a conductive substrate to obtain a surface modified conductive substrate;
e) drying the surface modified conductive substrate at a temperature range of 30 to 50° C. for a period between 30 min to 120 min to form a dried nanocomposite on the conductive substrate;
f) treating the dried nanocomposite on the conductive substrate with an anti-Ova solution;
g) washing the treated conductive substrate using the phosphate-buffered saline (PBS) solution to obtain a washed conductive substrate;
h) treating the washed conductive substrate with a bovine serum albumin solution to obtain a bovine serum albumin treated conductive substrate; and
i) washing the bovine serum albumin treated conductive substrate using the PBS solution to obtain the immunosensor electrode for detecting ovalbumin.

20. The method as claimed in claim 19, wherein step (a) comprises ultrasonicating 50 mg MXene nanosheets in 10 ml water to obtain a uniform MXene suspension.

21. The method as claimed in claim 19, wherein step (b) comprises sonicating the mixture for 15 minutes.

22. The method as claimed in claim 19, wherein step (b) comprises modifying a nanocellulose using a periodate oxidation method to obtain a dialdehyde nanocellulose.

23. The method as claimed in claim 22, wherein the periodate oxidation method comprises treating the nanocellulose with a periodate salt for 4-72 hours at a temperature range of 30 to 80° C. in absence of light.

24. The method as claimed in claim 23, wherein the periodate salt is sodium metaperiodate or sodium orthoperiodate.

25. The method as claimed in claim 19, wherein the suspension of zirconium oxide nanoparticles in step (c) is obtained by dispersing a nanostructured zirconium oxide powder in distilled water.

26. The method as claimed in claim 19, wherein the homogenous nanocomposite suspension in step (c) is subjected to further mixing and sonication for a period range of 30 min to 120 min.

27. The method as claimed in claim 19, wherein the conductive substrate is a glassy carbon electrode, indium tin oxide (ITO), Fluorine-doped tin oxide (FTO) or screen printed electrodes.

28. The method as claimed in claim 19, wherein step (f) is carried out by using 4 μl of 1 μg/ml of the anti-Ova solution.

29. The method as claimed in claim 19, wherein step (f) comprises incubating the treated conductive substrate with the anti-Ova solution at a temperature range of 25 to 35° C. for 1 hour.

30. The method as claimed in claim 19, wherein step (h) comprises dropping 10 μL of 0.1% bovine serum albumin solution onto the surface of the washed conductive substrate and incubating the bovine serum albumin treated conductive substrate at 25° C. for 45 min to block the non-reactive sites and to reduce non-specific interactions on the electrode surface.

31. A method of using an immunosensor electrode for detecting ovalbumin, the immunosensor electrode comprising a conductive nanocomposite formed on a surface of a conductive substrate and a biological recognition element bound onto the conductive nanocomposite wherein the biological recognition element is selective towards the egg protein, wherein the conductive nanocomposite comprising a cellulose derivative which is capable of binding with the biological recognition element wherein the biological recognition element having selectivity towards the egg protein, a nanostructured metal oxide which is capable of facilitating an electric conductivity of the immunosensor electrode and a two-dimensional conductive nanomaterial which is capable of connecting the cellulose derivative to the nanostructured metal oxide, wherein the conductive nanocomposite facilitates a measurement of the electric conductivity of the immunosensor electrode upon an interaction between the biological recognition element bound on the cellulose derivative with the egg protein, wherein the method comprises steps of:
a) extracting a food sample by incubating the food sample with a PBS solution at a temperature range of 30 to 60° C. for a period range of a period of 20 min to 24 hours to obtain a food sample mixture;
b) centrifuging the food sample mixture to obtain a supernatant;
c) diluting the supernatant using a second PBS solution with a ratio of range from 1:100 to 1:1000 to obtain a diluted supernatant solution;
d) incubating the diluted supernatant solution for a period of 15 min to 60 min with the immunosensor electrode;
e) detecting the ovalbumin by conducting an electrochemical measurement using the immunosensor electrode and an electrochemical workstation, wherein the electrochemical measurement is cyclic voltammetry (CV); and
f) detecting the presence of ovalbumin by conducting an electrochemical measurement using the immunosensor electrode and an electrochemical workstation, wherein the electrochemical measurement is differential pulse voltammetry (DPV)
wherein the immunosensor electrode is used as a working electrode in a three-electrode electrochemical workstation,
wherein the concentration of ovalbumin is determined by measuring the DPV using the immunosensor electrode,
wherein a low concentration of ovalbumin results in a high conductivity measured by the electrochemical workstation using the immunosensor electrode, and
wherein a high concentration of ovalbumin resulted in a low conductivity measured by the electrochemical workstation using the immunosensor electrode.

32. A method of using an immunosensor electrode for detecting ovalbumin, the method comprising steps of:
a) dispersing a MXene nanosheet into an aqueous solution to form a MXene suspension;
b) mixing the MXene suspension with a nanocellulose derivative to form a mixture;
c) adding a suspension of zirconium oxide nanoparticles into the mixture to form a nanocomposite homogeneous suspension;
d) drop-casting the nanocomposite homogeneous suspension onto a surface of a conductive substrate to obtain a surface modified conductive substrate;
e) drying the surface modified conductive substrate at a temperature range of 30 to 50° C. for a period between 30 min to 120 min to form a dried nanocomposite on the conductive substrate;
f) treating the dried nanocomposite on the conductive substrate with an anti-Ova solution;
g) washing the treated conductive substrate using the phosphate-buffered saline (PBS) solution to obtain a washed conductive substrate;
h) treating the washed conductive substrate with a bovine serum albumin solution to obtain a bovine serum albumin treated conductive substrate;
i) washing the bovine serum albumin treated conductive substrate using the PBS solution to obtain the immunosensor electrode for detecting ovalbumin;
j) extracting a food sample by incubating the food sample with a PBS solution at a temperature range of 30 to 60° C. for a period range of a period of 20 min to 24 hours to obtain a food sample mixture;
k) centrifuging the food sample mixture to obtain a supernatant;
l) diluting the supernatant using a second PBS solution with a ratio of range from 1:100 to 1:1000 to obtain a diluted supernatant solution;
m) incubating the diluted supernatant solution for a period of 15 min to 60 min with the immunosensor electrode;
n) detecting the ovalbumin by conducting an electrochemical measurement using the immunosensor electrode and an electrochemical workstation, wherein the electrochemical measurement is cyclic voltammetry (CV); and
o) detecting the presence of ovalbumin by conducting an electrochemical measurement using the immunosensor electrode and an electrochemical workstation, wherein the electrochemical measurement is differential pulse voltammetry (DPV),
wherein the immunosensor electrode is used as a working electrode in a three-electrode electrochemical workstation,
wherein the concentration of ovalbumin is determined by measuring the DPV using the immunosensor electrode,
wherein a low concentration of ovalbumin results in a high conductivity measured by the electrochemical workstation using the immunosensor electrode, and
wherein a high concentration of ovalbumin resulted in a low conductivity measured by the electrochemical workstation using the immunosensor electrode.
